(19) United States
(12) Patent Application Publication
(10) Pub. No.: US 2026/0191794 A1
(43) Pub. Date: Jul. 9, 2026

(54) METHOD FOR PREVENTING OR TREATING AGE RELATED CONDITIONS USING EXTRACELLULAR VESICLES DERIVED FROM A YOUNG MAMMAL

(71) Applicant: ExoMed Pty Ltd, Mudgee, New South Wales (AU)

(72) Inventor: Geoffrey KEIPERT, Mudgee, New South Wales (AU)

(21) Appl. No.: 19/132,320

(22) PCT Filed: Nov. 23, 2023
(86) PCT No.: PCT/AU2023/051198

§ 371 Date: May 22, 2025

(30) Foreign Application Priority Data
Nov. 23, 2022 (AU) 2022903549

(51) Int. Cl. A61K 9/50; A61K 9/00; A61P 1/16; A61P 13/12; A61P 25/28; A61P 39/00

(52) U.S. Cl. CPC A61K 9/5068; A61K 9/0053; A61K 9/5089; A61P 1/16; A61P 13/12; A61P 25/28; A61P 39/00

(57) ABSTRACT

The present disclosure relates to a method of treating, preventing or delaying an age-related condition or pathology in a mammal using extracellular vesicles (EVs) derived from a young mammal.

### CROSS-REFERENCE TO RELATED APPLICATIONS

The present application claims priority from Australian Provisional Patent Application No 2022903549 filed on 23 Nov. 2022, the content of which is incorporated herein by reference in its entirety.

### TECHNICAL FIELD

The present disclosure relates generally to oral compositions comprising extracellular vesicles (EVs) derived from young (e.g., juvenile) non-human animals, and methods of orally administering same to subjects in need thereof to treat, prevent or delay age-related conditions or pathologies.

### BACKGROUND

Aging is associated with the progressive degeneration of tissues that results in deterioration in the structure and function of organs within the body. It is characterized by a systemic decline in physical and cognitive function due to a deterioration in cellular mechanisms that maintain health and stress resistance, resulting in a decline in homeostasis and the capacity to respond to external stimuli. Although ageing is not considered a disease per se, it is associated with several chronic diseases such as cardiovascular disease, arthritis and cancer. The balance between the rate of damage and repair determines whether organ integrity is maintained and is regulated by both genetic and extra and intracellular molecular factors. Deterioration of the homeostatic mechanisms within the nervous, endocrine and immune systems drives the common features associated with ageing such as immunosenescence, inflammaging and cellular senescence. At present, there are no specific pharmaceutical interventions to treat, prevent or delay ageing and age-associated diseases.

Experiments with heterochronic parabiosis, heterochronic organ transplants and infusion of young plasma into old mice and rats has demonstrated that it is possible to reverse the ageing phenotype by exposing the tissues and organs of aged animals to a youthful environment. Whilst attempts to identify specific factors in young blood responsible for the rejuvenating effect have not been successful, some studies suggest that EVs may contribute to the rejuvenation process. Conversely, in other experiments, parabiosis and plasma injections have demonstrated a pro-ageing effect in young rodents with the introduction of an aged systemic milieu. It has therefore also been suggested that EVs circulating in disease states e.g., cancer and neurodegenerative disease, may promulgate the respective disease state when transferred to other mice. Further, senescent cell-derived EVs have also be shown to propagate an inflammatory state by spreading the senescence associated secretory phenotype (SASP) and increasing senescence in recipient tissues. These EVs increase over time in the aged EV pool. Senescent cells accumulate in organs with age and are known to contribute to tissue dysfunction. Thus, by virtue of their ability to induce physiological changes in cells, EVs have been shown to play a significant role in ageing and disease as well as rejuvenation. The fact that this can occur through adding youthful factors or other methods, such as exercise and caloric restriction, demonstrates that loss of the repair processes with age is not irreversible and suggests that they can be reactivated.

Most studies exploring the therapeutic effect of EVs have injected a single bolus of EVs into the animal that is likely to be supra-physiological, resulting in the bulk of EVs being directed to organs responsible for clearance before reaching other target cells. One recent study by Horvath et al., (2023) Geroscience demonstrates an epigenetic age reversal in rats via the intravenous administration of exosome-containing fraction derived from the plasma of young adult pigs. Similarly, WO2017/189842 describes experiments in which intraperitoneal injection of EVs derived from stem cells or serum of mice is used to treat age-related symptoms caused by stem cell dysfunction or increased senescence in a murine model. Sanz-Ros, et al., (2022) Sci Adv 8 (42): 1-18 demonstrates improvements in health of aging mice using intravenously administered small EVs derived from adipose mesenchymal stem cells (ADSCs) of young animals.

Notwithstanding the growing body of evidence that EV's can have a rejuvenating effect when administered parenterally in a laboratory setting, the repeated parenteral administration by intravenous and intraperitoneal routes is unlikely to be a practical long term strategy for delivery to humans and has the added disadvantage of potentially initiating an immune response, especially with xenogeneic EVs. As such, there remains a need for therapeutic compositions and methods for treatment, prevention or delay of ageing and age-associated conditions or pathologies using same, where the therapeutic that can be administered easily (e.g., self-administered) and is well-tolerated in the long term.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is based, inter alia, on the recognition by the inventor that EVs derived from young animals may exert a rejuvenating effect when administered parenterally and may therefore be useful for treating, preventing or delaying age-related conditions or pathologies. However, in order for EV's to become a feasible therapeutic strategy for treatment of age-related conditions and pathologies (or indeed any condition for that matter), the inventor recognised that it would be necessary to develop a safer, more convenient and less invasive method of administering a therapeutically effective dose of EVs to a subject; particularly for long-term treatment strategies. The inventor therefore set out to develop an oral formulation comprising EVs derived from young animals, as well as methods for treating, preventing or delaying ageing and age-associated conditions or pathologies by administering the formulation by the oral route. Whilst the literature recognised that EVs derived from young animals may possess rejuvenating properties when administered parenterally, to the inventor's knowledge there existed no evidence that delivery of EVs derived from young animals via the oral route could confer the same or a similar physiological effect as parenteral administration to aged animals, much less that oral administration of EVs would be well tolerated. In particular, it was unclear whether or not there would be sufficient uptake and bioavailability of EVs when delivered by the oral route, and whether any EV's delivered orally would retain their effectiveness and efficiency after travelling through the gastrointestinal tract. In this regard, there has been little progress in achieving systemic delivery of other intact proteins or nucleic acids via the oral route, and the pharmacodynamics and pharmacokinetics of orally-delivered EVs have not been well-studied or validated. The inventor therefore explored the rejuvenating potential of EVs derived from young animals (e.g., young mice and young cattle) when administered to aged mice orally. In doing so, the inventor has shown for the first time that treatment of aged mice with EVs derived from young animals, delivered via the oral route, is capable of improving a number key parameters associated with age-related diseases and pathologies, including cognitive function, physical performance, metabolic function, inflammation and oxidative stress. Moreover, the inventor has demonstrated that EVs derived from young cattle (i.e., calves) are well tolerated when administered orally to aged mice and that those EVs are capable of conferring the above-mentioned physiological improvements despite being of xenogeneic origin. Thus, the inventor has demonstrated for the first time, and unexpectedly, that EVs derived from young animals (e.g., juveniles) possess rejuvenating properties when delivered orally to aged animals, even when those EVs are of xenogeneic origin.

An advantage of the therapeutic approach developed herein is that it leverages oral tolerance mechanisms, akin to those with food proteins, to substantially lower the likelihood of a subject developing an allergic response. In particular, since all methods of purification of EVs have been shown to present with a degree of protein and lipoprotein contamination, repeated systemic injection a demonstrated in the studies to date presents a significant risk for the development of an antibody response to foreign proteins and a potential allergic reaction. This is an important consideration in designing an administration strategy since treatments aimed at rejuvenation or anti-aging using EVs will likely require frequent administration over a prolonged duration. Furthermore, zoonotic viruses, such as latent endogenous viruses in animal tissue, from pigs and cattle represent a potential hazard for systemic administration. This is expected to present a significant regulatory challenge for EVs sourced from animals and intended for systemic injection in humans. This challenge is mitigated by formulating the EVs for oral administration since plasma (or other tissue sources) can be manufactured to food-grade standards. Thus, the oral formulations of the disclosure provide a number of advantages.

The inventor also postulates that the ability to source EVs from various species, and from a species of animal which is different to that of the subject to be treated, presents certain advantages in terms of manufacture and sourcing of EVs. To date, the production of EVs has been confined to small-scale laboratory settings which has been recognized as an impediment to scaling up production for therapeutic applications. Consequently, the inventor recognized the need for a readily available and substantial source of EVs that allows for simple extraction from raw materials, be cost-efficient, and yield a satisfactory quantity of EVs. For example, by-product (e.g., blood, tissue and organs) from livestock slaughtered for human consumption may provide a readily-available source of EVs that can be processed and formulated for oral delivery to human and non-human subjects. The selected raw material supply chain combined with simplified extraction and minimal purification acceptable for oral administration bypasses the rigorous and costly regulatory standards for an injectable regenerative biotherapeutic. Thus, in addition to the therapeutic advantages identified by the inventor, the present disclosure provides a further and novel use for slaughterhouse waste.

Accordingly, in one example, the present disclosure provides an oral composition comprising a therapeutically effective amount of EVs derived from blood, a blood fraction and/or tissue of a young, non-human animal.

In one example, the young, non-human animal is of an age up to and including the age of sexual maturity. For example, the young, non-human animal may be an animal which has not yet reached sexual maturity. For example, the young, non-human animal may be a juvenile animal e.g., an animal which has not yet reached its adult form, sexual maturity and/or size.

In one example, the EVs may be derived from a livestock species. For example, the EVs may be derived from a mammalian livestock species. Exemplary non-human mammals may be selected from bovine, ovine, equine, caprine, porcine, camelid or cervine species. In one example, the EVs are derived from a bovine animal. In one example, the EVs are derived from an ovine animal. In one example, the EVs are derived from an equine animal. In one example, the EVs are derived from a caprine animal. In one example, the EVs are derived from a porcine animal. In one example, the EVs are derived from a camelid animal. In one example, the EVs are derived from a cervine animal. Alternatively, or in addition, the EVs may be derived from a non-mammalian livestock species e.g., poultry.

The EVs described herein may be selected from the group consisting of exosomes, microvesicles, membrane particles, membrane vesicles, exosome-like vesicles, extracellular vesicles, exovesicle and combinations thereof.

As described herein, the EVs may be derived from blood, a blood fraction and/or tissue of a young, non-human animal. In one example, the EVs are derived from whole blood. In one example, the EVs are derived from a blood fraction, such as serum or plasma. In one example, the EVs are derived from bone marrow. In other examples, the EVs are derived from tissue. For example, the tissue may be selected from adipose tissue, thymus, pancreas, lung, heart, liver, muscle, intestine and any combination thereof. In one particular example, the tissue is selected from adipose tissue, thymus and pancreas.

In some examples, the EVs are present in the oral composition described herein at a concentration which is higher than is present in the blood, blood fraction or tissue from which the EVs are derived.

In one example, the EVs have been purified. In accordance with this example, the EVs are purified from the blood, blood fraction or tissue from which they are derived (i.e., separated from other component parts of the blood, blood fraction or tissue respectively) and formulated into the oral composition.

In one example, the EVs are lyophilised. In another example, the EVs are spray-dried. In a further example, the EVs are gelated. In another example, the EVs are encapsulated. In some examples, the EVs are lyophilised or spray dried and then encapsulated.

In one examples, the EVs are provided in a liquid form.

In some examples, the EVs are provided within a capsule. That is, in some examples the oral composition is provided as a capsule comprising the EVs described herein.

The present disclosure also provides a method of preparing an oral composition as described herein, comprising:
a) obtaining blood, a blood fraction and/or tissue from a non-human mammal; and
b) isolating the EVs from one or more other components of the blood, blood fraction and/or tissue.

The EVs isolated from the blood, blood fraction and/or tissue may then be formulated in a form suitable for oral administration.

In some examples, the method preparing an oral composition as described herein further comprises performing one or more purification processes and/or one of more processes to concentrate the EVs, following isolation of the EVs from one or more other components of the blood, blood fraction and/or tissue. The one or more purification processes may be selected from size exclusion chromatography, ultracentrifugation, polymer and calcium ion precipitation, salt-induced precipitation and ultrafiltration. In one example, the method comprises concentrating the EVs by ultrafiltration and centrifugation. In some examples, the method of the disclosure comprises one or more purification processes and one of more processes to concentrate the EVs (e.g., relative to the concentration of EVs in the isolate obtained from the blood, blood fraction and/or tissue).

Alternatively, or in addition, the method described herein may further comprise lyophilizing and/or encapsulating the EVs. For example, the method may comprise lyophilizing and encapsulating the EVs. In accordance with an example in which the EVs are purified and/or concentrated, the lyophilisation and/or encapsulation steps may be performed on the purified and/or concentrated EVs.

The method of preparing an oral composition as described herein may further comprise formulating the EVs in a capsule which is suitable for oral administration.

The present disclosure also provides oral compositions when produced by the methods described herein.

The present disclosure also provides a method of treating, preventing or delaying an age-related condition or pathology in a subject, comprising orally administering to the subject the oral composition described herein.

The present disclosure also provides for the use of a composition described herein in the manufacture of a medicament for treating, preventing or delaying an age-related condition or pathology in a subject, wherein the medicament is an oral medicament.

As described herein, the age-related condition or pathology may be associated with a decline in one or more of cognitive function, physical performance and/or metabolic function.

In one example, the age-related condition or pathology is associated with a decline in cognitive function. For example, the decline in cognitive function may comprise a decline in one or more of alertness, executive function, memory, learning, visual-spacial processing utilizing the frontal cortex, thalamus, dorso-lateral prefrontal cortex and/or hippocampus, and/or olfactory sensitivity.

Alternatively, or in addition, the age-related condition or pathology is associated with a decline in physical performance. For example, the decline in physical performance may comprise a decline in one or more of musculoskeletal strength, motor skills and/or balance, and/or an increase in frailty.

Alternatively, or in addition, the age-related condition or pathology is associated with a decline in metabolic function. For example, the decline in metabolic function may comprise impaired glucose tolerance and/or insulin insensitivity.

In some examples, the age-related condition or pathology may be associated with an increase in inflammation and oxidative stress in the tissue of the subject. For example, inflammation and/or oxidative stress may be associated with impaired kidney function, acute kidney injury, chronic kidney disease, or acute and chronic liver injury in the subject. Accordingly, the method or use of the disclosure may be for treating, preventing or delaying an age-related condition or pathology selected from impaired kidney function, acute kidney injury, chronic kidney disease, or acute and chronic liver injury in a subject in need thereof. In one example, the impaired kidney function is due to an acute kidney injury. In another example, the impaired kidney function is due to a chronic kidney injury. In a one example, the impaired liver function is due to an acute liver injury. In another example, the impaired liver function is due to a chronic liver injury.

The present disclosure also provides a method of improving kidney function in a subject suffering from impaired kidney function, acute kidney injury or chronic kidney disease, comprising orally administering to the subject the oral composition described herein.

The present disclosure also provides for use of a composition described herein in the manufacture of a medicament for improving kidney function in a subject suffering from impaired kidney function, wherein improving kidney function in the subject comprises administering the medicament to the subject orally.

In one example, the impaired kidney function is due to an acute kidney injury.

Accordingly, the subject may be suffering from an acute kidney injury. In another example, the impaired kidney function is due to a chronic kidney injury. Accordingly, the subject may be suffering from a chronic kidney injury.

The present disclosure also provides a method of improving liver function in a subject suffering from impaired liver function, comprising orally administering to the subject the oral composition described herein.

The present disclosure also provides for use of a composition described herein in the manufacture of a medicament for improving liver function in a subject suffering from impaired liver function, wherein improving liver function in the subject comprises administering the medicament to the subject orally.

In one example, the impaired liver function is due to an acute liver injury. Accordingly, the subject may be suffering from an acute liver injury. In another example, the impaired liver function is due to a chronic liver injury. Accordingly, the subject may be suffering from a chronic liver injury.

### BRIEF DESCRIPTION OF THE DRAWINGS

The disclosure may be better understood by reference to one or more of these figures in combination with the detailed description of specific embodiments presented herein.

FIG. 1: Discrimination index scores recorded for mice in the different treatment groups performing the novel object recognition test (NORT).

FIG. 2: A. Mean escape latency time (secs) recorded for mice in the different treatment groups during the trial period of the Barnes Maze test. B. Number of correct attempts (nose pokes) recorded for mice finding the escape hole in the Maze.

FIG. 3: Assessment of fear-conditioned response in mice from each treatment group as measured by the percentage of time spent freezing.

FIG. 4: A. Olfactory sensitivity measured by total exploratory time (secs) of mice in the different treatment groups at each odorant dilution. B. Latency (secs) for mice in the different treatment groups to find buried food.

FIG. 5: Scores recorded for mice in the different treatment groups performing the tightrope test.

FIG. 6: A. Body mass (g) recorded for mice in the different treatment groups prior to performing the 4 limb hanging test. B. Hanging time (secs) measured for each mouse on the inverted screen.

FIG. 7: Frailty index score based on several parameters (as detailed in the assessment form) for mice in the difference treatment groups.

FIG. 8: A. Blood glucose levels (mmol/L) in mice injected with 20% glucose solution measured every 15 min over 120 min. B. Insulin sensitivity and glucose homeostasis as measure by area under curve (mmol/L×min) in mice from the different treatment groups.

FIG. 9: Measure of serum liver enzymes in mice from the different treatment groups to assess liver function following exposure to CCL4 (A. baseline levels of serum liver enzymes in untreated, young and old mice; B. alanine transaminase, ALT; C. aspartate transaminase, AST; D. alkaline phosphatase, ALP) E. Measure malondialdehyde (MDA) levels as a marker of oxidative stress in the liver of these mice. F. Measure of hydroxyproline levels as a marker of fibrosis in the liver of these mice.

FIG. 10: Measure of serum kidney parameters in mice from the different treatment groups to assess kidney function following exposure to haemoglobin (A. urea; B. creatinine). C. Measure of malondialdehyde (MDA) levels as a marker of oxidative stress in the kidney of these mice.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in genomics, immunology, molecular biology, immunohistochemistry, biochemistry, oncology, and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984), Methods In Enzymology (S. Colowick and N. Kaplan, eds., Academic Press, Inc.), whole of series, and Remington's Pharmaceutical Sciences, 16th Ed. Mack Publishing Company, 1980.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

Throughout this specification, the word “comprise’ or variations such as “comprises” or “comprising” will be understood to imply the inclusion of a stated element, integer or step, or group of elements, integers or steps, but not the exclusion of any other element, integer or step, or group of elements, integers or steps.

The term “about” is used herein to mean approximately. When the term “about” is used in conjunction with a numerical range, it modifies that range by extending the boundaries above and below the recited numerical values. In general, the term “about” is used herein to modify a numerical value, such as an amount of time, concentration, temperature etc., above and below the stated value by ±10%, more preferably ±5%, even more preferably ±1%, and still more preferably ±0.1% from the specified value as appropriate to perform the disclosed method.

### Oral Compositions

The present disclosure underscores the inventor's innovative approach in developing an oral therapeutic composition designed for treating, preventing and/or delaying age-related conditions, with a focus on ease of administration, sustained tolerability and therapeutic efficacy. Specifically, this approach addresses the uncertainties surrounding both the bioavailability and the therapeutic effectiveness of EVs when administered orally. Indeed, while the application of several orally-delivered EV compositions (for example, milk EVs and plant EVs) have been investigated, results pertaining to systemic absorption, biodistribution and bioavailability have been inconclusive.

As described herein, the inventor has shown for the first time that oral delivery of EVs derived from blood, a blood fraction and/or tissue of a young, non-human animal is capable of improving one or more parameters associated with age-related diseases and pathologies in aging animals, including, but not limited to, cognitive function, physical performance, metabolic function, inflammation and oxidative stress. Accordingly, the present disclosure provides oral compositions comprising a therapeutically effective amount of extracellular vesicles (EVs) derived from blood, a blood fraction and/or tissue of a young, non-human animal, and the use of said compositions to treat, prevent or delay age-related conditions or pathologies in a mammalian subject via the oral route.

The terms “extracellular vesicles”, “EVs” or “microvesicles” shall be understood to mean nano-sized membrane-bound structures having a lipid bilayer which are released by cells. EVs range in diameter from near the size of the smallest physically possible unilamellar liposome (around 20-30 nm) to as large as 1 micron or more. They are capable of carrying cargos comprised of proteins, nucleic acids, lipids, metabolites, and even organelles from the parent cell from which they are released. It is noted that the terms “extracellular vesicles” and “microvesicles” are used interchangeably herein to describe all cell-derived membrane-bound structures excluding apoptotic bodies.

A wide variety of EV subtypes have been proposed, defined variously by size, biogenesis pathway, cargo, cellular source, and function. There is also some heterogeneity in the nomenclature used to define various EV subtypes. It is contemplated that the oral composition of the disclosure may comprise EVs which are present in blood, blood fractions and/or tissues of young, non-human animals. Exemplary EV subtypes include, but are not limited to exosomes, microvesicles, oncosomes, ectosomes, prostasomes, matrix/calcifying vesicles, tolerosomes, cardiosomes, and vexosomes and any combinations thereof. Extracellular vesicles and their respective properties are discussed in Lotvall et al., (2014) Journal of Extracellular Vesicles 3:26913 and Zempleni et al., (2013) Nature Reviews Drug Discovery 12:347-357, the contents of which are incorporated by reference herein.

As described herein, EVs vary in size and a given sample of EVs will have an average diameter with individual vesicles varying within a range. EVs in the composition of the disclosure may range in size with a lower size limit of about 20-50 nanometers (nm) in diameter and an upper size limit of about 1 micron in diameter.

The EVs used to formulate the oral composition of the disclosure may be derived from blood, a blood fraction and/or tissue of a young, non-human animal. As used herein, the term “blood” shall be understood to include whole blood, which is composed of blood plasma, which is typically unclotted, and cellular components. The plasma typically represents about 50% to about 60% of the volume, and cellular components, i.e. erythrocytes (red blood cells, or RBCs), leucocytes (white blood cells, or WBCs), and thrombocytes (platelets), represent about 40% to about 50% of the volume. In some examples, the EVs within the oral composition of the disclosure are derived from whole blood. In other examples, the EVs in the oral composition are derived from a “blood fraction” or a component of whole blood. For example, EV of the disclosure may be derived from plasma, serum, red blood cells, platelets, fluids comprising any of these substances, and/or fluids derived from any of these substances. The terms “blood plasma” or “plasma” as used herein refer to the liquid fraction of blood and lymphatic fluid, which makes up about half of the volume of blood (e.g. about 50 to about 60 vol.-%). Plasma is devoid of cells, and unlike serum, has not clotted. It is a clear yellowish liquid comprising about 90 to about 95 vol.-% water. The terms “blood serum” or “serum” as used herein refer to the clear liquid that separates from blood when it is allowed to clot completely, and is therefore blood plasma from which in particular fibrinogen has been removed during clotting. Like plasma, serum is light yellow in colour.

Alternatively, or in addition, the EVs comprised within the composition of the disclosure may be derived from non-human animal tissue. In one example, the tissue is a blood-related tissue, such as bone marrow. The term “bone marrow” as used herein refers to the gelatinous tissue found in the hollow interior of mammalian and avian bones, particularly mammalian. In other examples, the tissue from which the EVs are derived is thymus, liver, spleen, lung, brain, kidneys, pancreas, gastrointestinal tract, small intestine, colon, stomach, heart and any combination thereof.

As described herein, the EVs comprised within the oral composition of the disclosure are derived from a non-human animal. In one example, the EVs may be derived from a livestock species. The term “livestock” as used herein, refers to domesticated animals raised in an agricultural setting for commercial purposes (e.g., meat, wool, milk, etc.). The livestock species may be a mammalian livestock species or an avian livestock species. However, in a particular example, the composition comprises EVs derived from a mammalian livestock species. Exemplary mammalian livestock species include bovine, ovine, equine, caprine, porcine, camelid or cervine species. Alternatively, or in addition, the EVs within a composition of the disclosure may be derived from a non-mammalian livestock species e.g., poultry.

The non-human animal from which the EV are derived is a young non-human animal. In this regard, the disclosure is based on the unexpected finding that EVs derived from young animals possess rejuvenating potential when administered orally to aged animals. The term “young” as used herein refers to an immature animal. For example, a young animal may be a juvenile animal, such as an animal which has not yet reached its adult form, sexual maturity and/or size. In some examples, a “young” animal shall be understood to refer to an animal which has not yet reached sexual maturity. Accordingly, in some examples, a young animal may be an animal which is of an age up to and including the age of sexual maturity.

As used herein, the term “sexual maturity” shall be understood to refer to the age or stage of development when an organism (e.g., an animal) can reproduce sexually. The skilled person will appreciate that the age of sexual maturity may differ between species, breed and/or gender, as well as between different individuals within any one or more of those categories.

In accordance with an example in which the animal is a bovine animal, the bovine animal may be less than about 24 months of age (e.g., less than about 18 months, or less than about 12 months of age, or less than about 9 months of age, or less than about 6 months of age). In one example, the bovine animal is less than about 24 months of age. For example, the bovine animal is between about 1 month to about 24 months of age (e.g. between about 1 month to about 6 months of age, or between about 1 month to about 12 months of age, or between about 1 month to about 18 months of age, or between about 6 months to about 12 months of age, or between about 6 months to about 18 months of age, or between about 12 months to about 18 months of age, or between about 12 months or about 24 months of age, or between about 18 months to about 24 months of age).

In accordance with an example in which the animal is an ovine animal, the ovine animal is less than about 15 months of age (e.g., less than about 9 months of age, or less than about 6 months of age, or less than about 4 months of age). In one example, the ovine animal is less than about 15 months of age. For example, the ovine animal is between about 1 month to about 15 months of age (e.g. between about 1 month to about 6 months of age, or between about 1 month to about 9 months of age, or between about 6 months to about 9 months of age, or between about 6 months to about 15 months of age, or between about 9 months to about 15 months of age).

In accordance with an example in which the animal is an equine animal, the equine animal is less than about 48 months or 60 months of age. However, in some examples, the equine animal is less than about 36 months of age (e.g., less than about 24 months of age, or less than about 18 months of age, or less than about 12 months of age). In one example, the equine animal is less than about 36 months of age. For example, the equine animal is between about 1 month to about 36 months of age (e.g. between about 1 month to about 6 months of age, or between about 6 months to about 12 months of age, or between about 6 months to about 18 months of age, or between about 6 months to about 24 months of age, or between about 6 months to about 36 months of age, or between about 12 months to about 24 months of age, or between about 12 months to about 24 months of age, or between about 12 months to about 36 months of age, or between about 18 months to about 24 months of age, or between about 18 months to about 36 months of age, or between about 24 months to about 36 months of age).

In accordance with an example in which the animal is an porcine animal, the porcine animal is less than 12 months of age (e.g., less than about 11 months of age, or less than about 10 month of age, or less than about 9 month of age, or less than about 8 month of age, less than about 7 months of age, or less than about 6 months of age, or less than about 5 months of age, or less than about 4 months of age). In one example, the porcine animal is less than about 12 months of age. In one example, the porcine animal is less than about 6 months of age. For example, the porcine animal may be between about 1 month to about 12 months of age (e.g. between about 1 month to about 3 months of age, or between about 1 month to about 6 months of age, or between about 1 month to about 9 months of age, or between about 3 months to about 9 months of age, or between about 6 months to about 12 months of age).

In accordance with an example in which the animal is a camelid animal, the camelid animal is less than about 36 months of age (e.g., less than about 24 months of age, or less than about 18 months of age, or less than about 12 months of age, or less than 6 months of age). In one example, the camelid animal is less than about 36 months of age. For example, the camelid animal is between about 1 month to about 36 months of age (e.g. between about 1 month to about 6 months of age, or between about 1 month to about 12 months of age, or between about 1 month to about 18 months of age, or between about 1 month to about 24 months of age, or between about 6 months to about 18 months of age, or between about 6 months to about 24 months of age, or between about 6 months to about 36 months of age, or between about 12 months to about 24 months of age, or between about 12 months to about 36 months of age, or between about 18 months to about 36 months of age, or between about 24 months to about 36 months of age).

In accordance with an example in which the animal is a cervine animal, the cervine animal may be less than about 18 months of age (e.g., less than about 12 months of age, or less than about 9 months of age, or less than about 6 months of age). In one particular example, the cervine animal is less than about 18 months of age. For example, the cervine animal is between about 1 month to about 18 months of age (e.g. between about 1 month to about 6 months of age, or between about 1 months to about 12 months of age, or between about 6 months to about 12 months of age, or between about 6 months to about 18 months of age, or between about 12 months to about 18 months of age).

The oral composition may comprise an amount of EVs from a young non-human animal which is therapeutically effective for treating, preventing or delaying an age-related condition or pathology in a subject. As used herein, the term “therapeutically effective amount” describes a quantity of a specified agent, such as EVs, sufficient to achieve a desired therapeutic, biological or physiological effect in a subject being treated with that agent. In the context of the composition of the disclosure, a “therapeutically effective” amount of EVs from a young non-human animal may be an amount sufficient to reduce, alleviate and/or prevent an age-related condition or pathology over the course of a treatment period. For example, an amount of EVs from a young non-human animal that is “therapeutically effective” may be amount that is effective to reduce, prevent or delay progression of an age-related condition or pathology associated with a decline in one or more of cognitive function, physical performance and/or metabolic function, or which is associated with oxidative stress and/or inflammation in tissue of a subject.

Ideally, a therapeutically effective amount of an agent is an amount sufficient to induce the desired result without causing a substantial cytotoxic effect in the subject. The effective amount of an agent useful for reducing, preventing and/or delaying the age-related conditions and pathologies described herein will be dependent on the subject being treated, the type and severity of any associated disease, disorder and/or condition (e.g., condition or pathology progression), and the manner of administration of the therapeutic composition.

The EVs in the oral composition of the disclosure may be present at a concentration which is higher than that present in the blood, blood fraction or tissue from which they are derived. In this regard, the EVs may have undergone one or more concentration or purification steps (e.g., to separate the EVs from other component parts of the blood, blood fraction or tissue respectively) prior to formulation of the oral composition.

The oral composition of the disclosure comprises at least 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, or 10¹², or 5×10¹² EVs derived from the blood, blood fraction or tissue of a young, non-human animal as described herein. In some examples, the oral composition may comprise between 10⁵, 10⁶, 10⁷, 10⁸, 10⁹, 10¹⁰, or 10¹¹ to 10⁶, 10⁷, 10⁸, 10⁹, 10¹⁰, 10¹¹, or 10¹², etc EVs which are derived from the blood, blood fraction or tissue of a young, non-human animal as described herein. In one particular example, the oral composition comprises at least 10⁶ or at least 10⁸ EVs derived from the blood, blood fraction or tissue of a young, non-human animal as described herein.

The EVs present in the oral composition may be lyophilized e.g., to improve stability of the EVs within the composition. As used herein, the term “lyophilised” shall be understood to mean that the EVs have been subjected to a lyophilization (i.e., freeze-drying) step. Thus, an EV preparation which is lyophilised can refer to a dry EV preparation or to a preparation of EV that has been subjected to lyophilization and subsequently been resuspended in an aqueous solution.

In some examples, the EVs present in the oral composition may be spray-dried. As used herein, the term “spray-dried” shall be understood to mean that the EVs have been transformed from a fluid state into a dried powder form by spraying the liquid feed into a hot drying medium.

In other examples, the EVs present in the oral composition may be gelated. As used herein, the term “gelated” shall be understood to mean that the EVs have been processed to form a three-dimensional network and have thus transited from a liquid composition to a viscous composition.

The oral composition of the disclosure may be formulated and provided in a solid or liquid form.

Solid dosage forms for oral administration include capsules, tablets, pills, powders, and granules. In such solid dosage forms, dry lyophilised EVs may be mixed with at least one inert, pharmaceutically acceptable excipient or carrier such as sodium citrate, dicalcium phosphate, or casein/caseinates and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Liquid forms of the oral composition include, without limitation, sterile solutions and suspensions. For example, the oral composition may comprise the EVs described herein formulated with a pharmaceutically and/or nutritionally acceptable carrier for administration to a mammal. Examples of such carriers include, without limitation, sterile aqueous or non-aqueous solutions, solvents, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other carriers used for oral administration.

The term “pharmaceutically and/or nutritionally acceptable carrier” refers to a non-toxic carrier or vehicle that does not destroy the pharmacological activity of the compound (or extracellular vesicles, as the case may be) with which it is formulated.

### Preparation of Oral Composition Comprising EVs

The present disclosure provides a method of preparing a composition described herein, comprising:
a). obtaining blood, a blood fraction and/or tissue from a young, non-human mammal; and
b). isolating the EVs from one or more other components of the blood, blood fraction and/or tissue.

The blood, a blood fraction and/or tissue of a young, non-human animal may be obtained from any suitable source prior to isolating EVs therefrom. In one example, the EVs may be isolated from blood, a blood fraction and/or tissue of a young livestock species. In this regard, by-product such as blood, tissue and organs from livestock slaughtered for human consumption may provide a readily-available source of materials from which EVs can be isolated and formulated for oral delivery to human and non-human subjects. Thus, in addition to the therapeutic advantages identified by the inventor, the present disclosure contemplates that the compositions and processes of producing same as described herein may provide a novel use for slaughterhouse waste. In one example, the livestock species may be a mammalian livestock species. Exemplary mammalian livestock species include, but are not limited to, bovine, ovine, equine, caprine, porcine or camelid species. In one example, the EVs are isolated from blood, a blood fraction and/or tissue of a young bovine animal. In one example, the EVs isolated from blood, a blood fraction and/or tissue of a young ovine animal. In one example, the EVs are isolated from blood, a blood fraction and/or tissue of a young equine animal. In one example, the EVs are isolated from blood, a blood fraction and/or tissue of a young caprine animal. In one example, the EVs are isolated from blood, a blood fraction and/or tissue of a young porcine animal. In one example, the EVs are isolated from blood, a blood fraction and/or tissue of a young camelid animal. Alternatively, or in addition, the EVs isolated from blood, a blood fraction and/or tissue of a young non-mammalian livestock species e.g., poultry.

As described herein, the non-human animal from which the blood, blood fraction and/or tissue is obtained is a young non-human animal. In this regard, the disclosure is based on the principle that EVs derived from blood, blood fraction and/or tissue obtained from young animals may possess rejuvenating potential or properties, and the unexpected finding by the inventor that these rejuvenating properties can confer an anti-aging effect (e.g., treat, prevent or delay an age-related condition or pathology) when administered orally to aged animals. A young, non-human animal from which the blood, blood fraction and/or tissue is obtained may be an immature animal. For example, a young animal may be a juvenile animal, such as an animal which has not yet reached its adult form, sexual maturity and/or size. In some examples, a young, non-human animal may be an animal which has not yet reached sexual maturity. Accordingly, in some examples, a young animal from which the blood, blood fraction and/or tissue is obtained may be an animal which is of an age up to and including the age of sexual maturity.

Exemplary young, non-human animals from which the blood, blood fraction and/or tissue may be obtained for isolation of EVs are described herein and shall be taken to apply mutatis mutandis to each and every example describing methods of producing oral compositions of the disclosure.

In one example, the method comprises obtaining whole blood from a non-human animal and isolating EVs therefrom. In one example, the method comprises obtaining a blood fraction, such as serum or plasma, from a non-human animal and isolating EVs therefrom. For example, EV of the disclosure may be isolated from plasma, serum, red blood cells, platelets, fluids comprising any of these substances, and/or fluids derived from any of these substances. In another example, the method comprises obtaining tissue from a non-human animal and isolating EVs therefrom. Suitable tissues include, but are not limited to, adipose tissue, bone marrow, thymus, liver, spleen, lung, brain, kidneys, pancreas, gastrointestinal tract, small intestine, colon, stomach, heart and any combination thereof.

As used herein, the term “isolating” refers to a process whereby the EVs are removed from their natural state e.g., blood, blood fraction and/or tissue. Accordingly, “isolated” EVs may be substantially or essentially free from components that normally accompany the EVs in their natural state (e.g., cells and/or cell debris).

The process of isolating (or harvesting) EVs from one or more other components of the blood, blood fraction and/or tissue may result in:
(i) the EVs having a greater concentration in the isolated form compared to the starting material (e.g., concentrating),
(ii) the removal of any amount or any type of impurities from the starting material (e.g., purifying),
(iii) an increase in the ratio of the amount of the EV relative to the amount of any undesired component in the starting material (e.g., enriching),
(iv) any artificial process for removing EVs from their natural source or location;
(v) any artificial process for separating EVs from at least one other component with which it is normally associated (e.g., purifying), or
(vi) any combination of (i), (ii), (iii), (iv) or (v).

In some examples, the step of isolating the EVs from blood, blood fraction and/or tissue purifies the EVs. However, in other examples, one or more additional purification steps may be performed. As used herein, the term “purifying” “purify”, “purification” or similar refers to a process for removing at least one impurity or contaminant from a starting material. For example, purifying a molecule of interest (e.g., an EV) from a starting material refers to a process for removing at least one impurity from the starting material to produce a relatively more pure form of the molecule of interest.

The EVs may be isolated and/or purified by any means known in the art, including centrifugation (e.g., differential ultracentrifugation), size exclusion filtration, size exclusion chromatography, affinity chromatography, density gradient centrifugation, immunoaffinity capture, polymer-based precipitation, calcium ion precipitation, salt-induced precipitation and by using microfluidics. In some examples, the isolation and/or purification of the EVs includes centrifugation of the cells and/or media conditioned by the cells. In some examples, ultracentrifugation is used. In some examples, the isolation and/or purification of the EVs is performed via size-exclusion filtration. In some examples, the isolation and/or purification of the EVs from a population of cells includes use of discontinuous density gradients, immunoaffinity, ultrafiltration and/or high performance liquid chromatography (HPLC).

In some examples, differential ultracentrifugation may be used tp isolate and/or purify EVs from cells or tissue, including centrifugal force from at least 1000×g, 2000×g, 3000×g, 4000×g, 5000×g, 6000×g, 7000×g, 8000×g, or 9000×g, to 2000×g, 3000×g, 4000×g, 5000×g, 6000×g, 7000×g, 8000×g, 9000×g, 10,000×g, or larger to separate larger-sized particles from the EVs derived from the cells or tissue.

In some examples, isolation and/or purification of EVs from a population of cells includes use of filtration or ultrafiltration. In certain examples, a size exclusion membrane with different pore sizes is used. For example, a size exclusion membrane can include use of a filter with a pore size of at least 0.1, 0.5 μm, 1.0 μm, 2.5 μm, 5 μm, to 0.5 μm, 1.0 μm, 2.5 μm, 5 μm, or larger. In some examples, the pore size is about 0.2 μm. In some examples, filtration or ultrafiltration includes size exclusion ranging from 0.1 kDa, 0.5 kDa, 1 kDa, 2 kDa, 5 kDa, 10 kDa, 25 kDa, 50 kDa, 100 kDa, or 250 kDa to 0.5 kDa, 1 kDa, 2 kDa, 5 kDa, 10 kDa, 25 kDa, 50 kDa, 100 kDa, 250 kDa, 500 kDa, or more.

In some examples, isolated and/or purified EVs are filter sterilized e.g., with a 0.22 μm microbial exclusion filter. In some examples, isolated and/or purified EVs are filtered using a 0.45 μm to remove cellular debris.

In some example, the isolation of EVs from a population of cells includes use of tangential flow filtration (TFF) systems to purify and/or concentrate the EV fractions. In other examples, the preparation of EVs from a population of cells includes use of (HPLC) to purify EVs to homogeneously sized particles. In other examples, density gradients may be used, such as centrifugation in a sucrose density gradient or application of a discrete sugar cushion in preparation.

In some examples, the preparation of EVs from a population of cells may include use of a precipitation reagent. For example, a precipitation reagent, such as EXOQUICK®, protamine, sodium acetate, magnesium sulfate, ammonium sulfate, sodium chloride, calcium chloride, potassium carbonate, and calcium sulfate or a combination thereof can be added to a solution containing a population of cells to quickly and rapidly precipitate a population of exosomes. In some examples, the preparation of EVs from a population of cells includes use of volume-excluding polymers (e.g., polyethylene glycols (PEGs)). In yet other examples, the preparation of EVs from a population of cells includes use of flow field-flow fractionation (FIFFF), an elution-based technique.

In some examples, an agent that causes aggregation and/or precipitation of the EVs may be added to the preparation prior to or after purification.

Any one or more of the processes for isolating or purifying the EVs may also be used for concentrating the EVs. That is, to increase the concentration of the EVs in the oral composition. Accordingly, in some examples, the method of the disclosure comprises a concentrating step so that the concentration of EVs in the oral composition is higher than what would be present in the blood, blood fraction or tissue from which they are derived.

The identity, purity, and integrity of the isolated and/or purified EVs can be confirmed using any number of methods known in the art, for example, western blot, nanoparticle tracking analysis, enzyme-linked immunosorbent assay (ELISA), flow cytometry and transmission electron microscopy. Absence of aggregation and EV purity can be assessed as recommended by the International Society for Extracellular Vesicles (Lotvall J, et al. Minimal experimental requirements for definition of extracellular vesicles and their functions: a position statement from the International Society for Extracellular Vesicles. J Extracell Vesicles 2014; 3:26913).

As described herein, the oral formulation of the disclosure comprises a therapeutically effective amount of EVs derived from the blood, blood fraction or tissue of a young, non-human animal. That is, a quantity or amount of the EVs sufficient to achieve a rejuvenating or anti-aging effect on a subject to whom the oral composition is administered. Accordingly, the method of the disclosure may comprise the step of formulating specific dosage forms of the oral composition comprising a therapeutically effective amount of the EVs isolated from the blood, blood fraction or tissue of a young, non-human animal. For example, the method may comprise formulating the oral composition such that a dosage form comprises at least 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, or 10¹², or 5×10¹² of the EVs isolated from the blood, blood fraction or tissue of a young, non-human animal as described herein. In some examples, the method may comprise producing a dosage form of the oral composition comprising between 10⁵, 10⁶, 10⁷, 10⁸, 10⁹, 10¹⁰, or 10¹¹ to 10⁶, 10⁷, 10⁸, 10⁹, 10¹⁰, 10¹¹, or 10¹², etc of the EVs isolated from the blood, blood fraction or tissue of a young, non-human animal as described herein. In one particular example, the oral composition produced by the method of the disclosure may comprise at least 10⁶ or at least 10⁸ of the EVs which have been isolated from the blood, blood fraction or tissue of a young, non-human animal as described herein.

As described herein, the oral composition of the disclosure may comprise EVs in a lyophilised spray-dried, or gelated form. Accordingly, the method for preparing the oral composition may further comprise the step of lyophilizing the EVs. “Lyophilizing” or “lyophilisation” refers to a drying method in which a aqueous-containing sample is frozen and placed under reduced pressure to sublimate and remove water in the sample and is placed in long-term storage. Protective agents of lyophilized EVs comprise methionine, mannitol and trehalose, and may be used in the form of an aqueous solution comprising ascorbic acid and retinol. The aqueous solution may be water for injection, physiological saline, phosphate buffer, purified water, or deionized water.

In accordance with examples in which the oral composition of the disclosure comprises spray dried EVs, the method for preparing the oral composition may comprise the step of spray-drying the EVs. Methods for spray drying are known in the art. Spray drying methods generally involve a process of transforming a material from a fluid state into a dried state by spraying the liquid feed into a hot drying medium. The resulting product is, in general and most preferably, a powder. In some examples, the powder may exhibit a degree of granulation and/or agglomeration.

In accordance with examples in which the oral composition of the disclosure comprises gelated EVs, the method for preparing the oral composition may comprise the step of gelation. Methods for gelation are known in the art and generally involves the formation of a three-dimensional network via cross-linking of branched polymers-referred to as gelling agents. Using these gelling agents, a viscous EV composition, or gel, may be formed from a liquid composition.

Where the EV composition is lyophilised or spray-dried, the composition may be retained as a dry EV preparation prior to formulation of the oral composition or resuspended in an aqueous solution prior to formulation of the oral composition. Accordingly, the oral composition produced by the method of the disclosure may be solid or liquid form.

Solid dosage forms of the oral composition which may be produced include capsules, tablets, pills, powders, and granules. In such solid dosage forms, dry lyophilised EVs may be mixed with at least one inert, pharmaceutically acceptable excipient or carrier such as sodium citrate, dicalcium phosphate or casein/caseinates and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Liquid forms of the oral composition which may be produced include, without limitation, sterile solutions and suspensions. For example, the oral composition may comprise the EVs described herein formulated with a pharmaceutically and/or nutritionally acceptable carrier for administration to a mammal. Examples of such carriers include, without limitation, sterile aqueous or non-aqueous solutions, solvents, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other carriers used for oral administration.

The term “pharmaceutically and/or nutritionally acceptable carrier” refers to a non-toxic carrier or vehicle that does not destroy the pharmacological activity of the compound (or extracellular vesicles, as the case may be) with which it is formulated.

### Methods of Use

Aging is a physiological process mediated by numerous biological and genetic pathways, which are directly linked to lifespan and are a driving force for all age-related diseases. It is the irreversibly progressive decline of physiological, physical and cognitive function, which eventually leads to a myriad of age-related diseases, such as cardiovascular diseases, musculoskeletal disorders, neurodegenerative diseases, organ dysfunction, diabetes and dementia. The present disclosure is based, inter alia, on the unexpected finding by the inventor that treatment of mature, aging animals via the oral route using an EV preparation isolated from blood obtained from young animals, is capable of improving a number key parameters associated with age-related diseases and pathologies, including cognitive function, physical performance, metabolic function, inflammation and oxidative stress. Accordingly, the present disclosure contemplates the use of the oral compositions described herein for prophylactic, preventative and/or therapeutic treatment of age-related conditions and/or pathologies in mammals.

As used herein, the terms “treating,” “treat,” or “treatment” includes (i) preventing a pathologic condition (e.g., fever or inflammation) from occurring (e.g., prophylaxis), (ii) inhibiting the pathologic condition (e.g., fever or inflammation) or arresting its development, and (iii) relieving the pathologic condition (e.g., relieving the symptoms associated with fever or inflammation).

In one example, the present disclosure provides a method of treating, preventing or delaying an age-related condition or pathology in a subject, comprising orally administering to the subject the oral composition as described herein.

In another example, present disclosure provides for the use of a composition described herein in the manufacture of a medicament for treating, preventing or delaying an age-related condition or pathology in a subject, wherein the medicament is formulated for oral administration.

The age-related condition or pathology may be associated with a decline in one or more of cognitive function, physical performance and/or metabolic function.

In one example, the subject to whom the oral composition or medicament is to be administered is suffering from or predisposed to an age-related condition or pathology associated with a decline in cognitive function of the subject. For example, the decline in cognitive function may comprise a decline in one or more of alertness, executive function, memory, learning, visual-spacial processing utilizing the frontal cortex, thalamus, dorso-lateral prefrontal cortex and/or hippocampus, and/or olfactory sensitivity. In some examples, administration of the oral composition or medicament to the subject may enhance neurological processes and/or cognitive function of the subject compared to a corresponding subject not administered the oral composition or medicament. For example, administration of the oral composition or medicament of the disclosure to the subject may result in an improvement in cognitive function in the subject as compared to a corresponding subject not administered the oral composition or medicament, wherein the improvement in cognitive function comprises one or more of an improvement in recognition memory, enhanced learning and memory based on spatial/visual cues, improved ability to learn and remember an association between environmental cues and aversive experiences, and increased olfactory sensitivity compared to a corresponding subject not administered the oral composition or medicament.

In another example, the subject to whom the oral composition or medicament is to be administered is suffering from or predisposed to an age-related condition or pathology associated with a decline in physical performance. For example, the decline in physical performance may comprise a decline in one or more of musculoskeletal strength, motor skills and/or balance, and/or an increase in frailty. In some examples, administration of the oral composition or medicament to the subject may enhance physical performance or muscle strength of the subject compared to a corresponding subject not administered the oral composition or medicament. For example, administration of the oral composition or medicament of the disclosure to the subject may result in an improvement in physical performance of the subject as compared to a corresponding subject not administered the oral composition or medicament, wherein the improvement in physical performance comprises one or more of an increase in strength, an increase in agility and/or a decrease in frailty compared to a corresponding subject not administered the oral composition or medicament.

In yet another example, the subject to whom the oral composition or medicament is to be administered is suffering from or predisposed to an age-related condition or pathology associated with decline in metabolic function. For example, the decline in metabolic function may comprise impaired glucose tolerance and/or insulin insensitivity. Accordingly, in some examples, administration of the oral composition or medicament to the subject may enhance one or more metabolic functions in the cells of the subject compared to a corresponding subject not administered the oral composition or medicament. For example, administration of the oral composition or medicament of the disclosure to the subject may result in an improvement in metabolic function of cell in the subject as compared to a corresponding subject not administered the oral composition or medicament, wherein the improvement in metabolic function comprises one or more of an an increase in glucose sensitivity and/or improvement in glucose homeostasis compared to a corresponding subject not administered the oral composition or medicament.

In yet another example, the subject to whom the oral composition or medicament is to be administered is suffering from or predisposed to an age-related condition or pathology associated with a decline in their immune system. Accordingly, in some examples, administration of the oral composition or medicament to the subject may enhance the immune system or response of the subject compared to a corresponding subject not administered the oral composition or medicament.

The inventor has also provided evidence that oral delivery of the composition or medicament described herein may be useful in the treatment of an age-related condition or pathology associated with inflammation and/or oxidative stress. In this regard, the inventor has shown that treatment of aged mice with the oral composition of the disclosure was able to reduce markers of inflammation and/or oxidative stress in the liver and kidneys of aged mice. Thus, in some examples, the age-related condition or pathology to be treatment may be associated with an increase in inflammation and oxidative stress in the tissue (e.g., kidney or liver) of the subject. For example, inflammation and/or oxidative stress may be associated with impaired kidney function, acute kidney injury, chronic kidney disease, or acute and chronic liver injury in the subject.

Accordingly, in one example, the present disclosure provides a method of improving kidney function in a subject suffering from impaired kidney function, acute kidney injury or chronic kidney disease, comprising orally administering to the subject the oral composition described herein.

The present disclosure also provides for use of a composition described herein in the manufacture of a medicament for improving kidney function in a subject suffering from impaired kidney function, wherein improving kidney function in the subject comprises administering the medicament to the subject orally.

Improved kidney function may comprise faster resolution of an acute insult to the kidney in a subject who received an acute kidney injury, or an improvement in kidney health in a subject suffering from a chronic kidney injury. Resolution of an acute insult to the kidney or overall kidney health may be determined by any means known in the art, such as e.g., by measuring levels of serum urea and/or creatinine in the subject following the acute kidney injury (e.g., 24 hrs after injury, or 48 hrs after injury, or 72 hr after injury etc). In one example, administration of the oral composition or medicament of the disclosure to a subject who has received an acute kidney injury may result in faster resolution of impaired kidney function as compared to a corresponding subject who has received an acute kidney injury but not been administered the oral composition or medicament. For example, faster resolution of the impaired kidney function may be determined by a decrease in serum urea and/or creatinine at least 24 hr (e.g., at least 48 hr) following insult to the kidneys relative of to the level serum urea and/or creatinine in the corresponding subject who has not been administered the oral composition or medicament. In one example, the improved kidney function may comprise faster resolution of an acute insult to the kidney in a subject who received an acute kidney injury. The level of resolution of an acute insult to the kidney may be determined by any means known in the art, such as e.g., by measuring levels of serum urea and/or creatinine and/or reactive oxygen species (ROS) and/or reactive nitrogen species (RNS) in the subject following the acute kidney injury (e.g., 24 hrs after injury, or 48 hrs after injury, or 72 hr after injury etc). In this regard, increased and/or sustained levels of serum urea, serum creatinine, ROS and/or RNS is indicative of kidney insult and associated impaired kidney function. Conversely, a decrease in one or more of serum urea, serum creatinine, ROS and/or RNS in a subject following acute kidney injury (e.g., 24 hrs after injury, or 48 hrs after injury, or 72 hr after injury etc) is indicative of improved kidney function an resolution of the kidney insult.

In one example, the impaired kidney function is due to an acute kidney injury. Accordingly, the subject may be suffering from an acute kidney injury. An acute kidney injury typically develops within hours or days and could be caused by physical trauma, acute intoxications, as part of multi-organ failure or various other diseases such as infections. The acute kidney injury could be pre-renal failure caused by haemorrhage, dehydration, osmotic diuresis, excessive diuretic use, sepsis, anaphylaxis, pancreatitis, afferent arteriolar vasoconstriction caused by NSAIDS, ACE inhibitors and other vasoconstrictors. The acute kidney injury could also be intrinsic-renal failure caused by primary or secondary glomerulonephritis (infective, autoimmune, inflammatory), ischaemia, nephrotoxins or sepsis. The acute kidney injury could also be post-renal failure caused by kidney stones or malignancies, bladder dysfunction, prostatic enlargement, uterine disease or obstructed catheters.

In another example, the impaired kidney function is due to a chronic kidney injury. Chronic kidney diseases typically develops over years and causes irreversible damage. Causes may include chronic bacterial infections leading to inflammation of the kidneys, cystic kidneys, various autoimmune diseases, hypertension, diabetes, prolonged renal obstruction, chronic exposure to toxic chemicals, toxins or drugs, nephrosclerosis, interstitial nephritis or pyelonephritis. Accordingly, the subject to be treatment may be suffering from a chronic kidney disease.

The present disclosure also provides a method of improving liver function in a subject suffering from impaired liver function, comprising orally administering to the subject the oral composition described herein.

The present disclosure also provides for use of a composition described herein in the manufacture of a medicament for improving liver function in a subject suffering from impaired liver function, wherein improving liver function in the subject comprises administering the medicament to the subject orally

The degree of impaired liver function and any improvement in liver function following treatment may be determined by means known in the art e.g., such as by measuring levels of biomarkers associated with hepatic inflammation (e.g., serum aminotransferases (ALT, AST) and/or alkaline phosphatase (ALP)), oxidative stress (e.g., malondialdehyde (MDA)) and/or fibrosis (e.g., hydroxyproline). In this regard, elevated levels of those biomarkers is indicative of liver injury and/or impaired liver function. As described herein, treatment of a subject suffering from a liver injury (acute or chronic) by administering the oral composition or medicament of the disclosure may result in a reduction in a level of one or more of ALT, AST, ALP, MDA and/or hydroxproline in the subject e.g., compared to a corresponding subject who is suffering from a liver injury (acute or chronic) but not been administered the oral composition or medicament.

In some examples, the impaired liver function may the result of an acute liver injury. An acute liver injury may be caused by drugs such as acetaminophen, NSAIDs, isoniazid and halothane, or toxins such as amanita phalloides or compounds derived from herbal medicines, viral infections such as hepatitis A, B, E or herpes simplex virus, vascular problems caused by shock, heat stroke or infiltrating malignancies, metabolic dysfunction such as acute fatty liver or alpha-1 antitrypsin deficiency.

In other examples, the impaired liver function may the result of a chronic liver disease. Chronic liver diseases may be caused by hemochromatosis, ischemia, sepsis, drugs, alcohol, viral infections with hepatitis A, B, or C, cirrhosis, fibrosis, cancer, non-alcoholic steatohepatitis or autoimmune hepatitis.

As used herein, “treating”, “treat” or “treatment” refers to a therapeutic intervention, course of action or protocol that at least ameliorates a symptom of such a disease, disorder or condition after said disease, disorder or condition and/or its symptoms have at least started to develop. As used herein, “preventing”, “prevent” or “prevention” refers to therapeutic intervention, course of action or protocol initiated prior to the onset of an age-related condition or pathology thereof so as to prevent, inhibit or delay the development or progression of such conditions, pathologies or a symptom thereof.

In accordance with the above-mentioned uses, the oral compositions of the disclosure may be administered to any animal. For example, the animal (or subject) to which the oral composition is administered may be a mammal, avian, chordate, amphibian or reptile. In one example, the animal is a mammal. Exemplary mammalian subjects include but are not limited to human, primate, livestock (e.g. sheep, cow, chicken, horse, camelid, goats, donkey, pig), companion animals (e.g. dogs, cats, horse), laboratory test animals (e.g. mice, rabbits, rats, guinea pigs, hamsters), captive wild animal (e.g. fox, deer). In one particular example, the subject to which the oral composition is orally administered is a human.

The subject to which the oral composition is orally administered is preferably a mature subject e.g., sexually mature subject. In some examples, the subject is suffering from one or more of the age-related conditions or pathologies described herein. In other examples, the subject is not suffering from one or more of the age-related conditions or pathologies described herein, but wishes to delay the aging process and prevent or delay the onset of age-related conditions or pathologies described herein.

The composition of the disclosure may be orally administered to a subject in need thereof in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried corn starch.

It would be understood that a specific dosage and treatment regimen for any particular patient will depend upon a variety of factors, including the age, body weight, general health, sex, diet, time of administration, rate of excretion, drug combination, and the judgment of the treating physician and the severity of the particular age-related condition or pathology being treated. Furthermore, the specific dosage may be varied over time as necessary to maintain efficacy and/or minimise any adverse events.

The oral composition of the disclosure may be administered in multiple doses at different time points with defined intervals. For example, the oral composition may be administered to a subject on a daily basis, or every 2 days, or every 3 days, or every 4 days, or every 5 days, or every 6 days, or on a weekly basis, or on a fortnightly basis or on a monthly basis, for example.

The frequency of treatment may be adjusted as necessary to ensure efficacy and/or minimise adverse events. In the case of a subject that is not adequately responding to treatment, multiple doses in a week may be administered. Alternatively, or in addition, increasing doses may be administered

### EXAMPLES

### Example 1. Assessing Cognitive Function in Aged Mice Treated with EVs Derived from Young and Old Bovine Animals

In this example, the inventor assessed whether treatment with EVs derived from young bovine animals improved cognitive function in age mice as compared to those treated with EVs derived from old bovine animals.

Cognitive abilities encompasses attention/alertness, executive function, memory and visual-spacial processing utilizing frontal cortex, thalamus, dorso-lateral prefrontal cortex and hippocampus. Function of these areas are known to decline with age. When evaluating age sensitivity for memory and learning, it is important to select tests that avoid food restriction or significant stressors that could impact on the ability of older animals to perform the tasks and confound the result. The novel objection recognition test (NORT), Barnes Maze test, fear recognition response test and olfactory test were employed here to assess cognitive function in aged mice treated with EVs derived from either young or old bovine animals.

### Materials and Methods

EV Extraction from Bovine Animals

Blood was collected from young (<12 month old weaner calves) and old bovine animals (8-9 years) by intravenous venepuncture into serum collection bags. Bags were centrifuged at 3000×g for 10 min at 4° C. to remove cellular debris and sediment erythrocytes. Supernatant was then centrifuged at 12,000×g for 45 min at 4° C. to remove apoptotic bodies and clarified using a 0.45 μm filter.

To reduce the level of protein contamination and impurities smaller than 20 nm (i.e., smaller than exosomes), the flowthrough was then subjected to filtration using a 100-300 kDa membrane. Depending on volumes required, this can be performed using a syringe filter, centrifugal filter or tangential flow filtration. The filtrate was then concentrated in a swinging bucket rotor centrifuge with the Jumbosep centrifugal filter (Pall, NY, USA). To reduce the risk of EV aggregation during concentration, the 0.45 μm filtered plasma was diluted 1:1 with 50 nM trehalose solution prior to concentration. After concentration, the presence of EVs was determined using the ExoELISA CD63 detection kit (Systems Biosciences, CA, USA). The EV concentrate was then resuspended in a 50 nM trehalose solution containing 8% reconstituted micellar casein to make up a 300 ul dose for oral gavage in mice.

### Treatment Groups

Aged wild type mice (22-24 months old) were divided into 2 groups and treated on alternate days with an EV concentrate. A minimum of 300 μl of the concentrate was administered every other day in the morning to the mice by oral gavage after withholding food overnight. Food was then re-introduced one hour later.

Aged wild type mice were divided into 2 groups:
1. (EV+) that were dosed orally with EVs sourced from young cattle.
2. (EV−) that were dosed orally with EVs sourced from old bovine animals.

Young wild type mice (3-4 months old) were used as a control for test parameters. The mice were treated for a 30 day period before performing any tests.

### NORT

The NORT assesses primary visual learning and is used to evaluate cognition, particularly recognition memory. The test is designed to assess short- or long-term recognition memory in mice and rats and relies on the innate preference of rodents for novelty. Aged mice have been shown to exhibit deficiencies in short term memory that affects their ability to recognize different objects. Performance is recorded by calculating a discrimination index that is based on the different times spent exploring the familiar and novel objects.

### Barnes Maze Test

The Barnes Maze assess hippocampal function and tests for impaired spatial memory. It evaluates learning and memory based primarily on spatial/visual cues. The test utilizes the natural tendency of a mouse to escape a brightly lit, exposed area. Performance measures latency and number of errors made by the mouse in the maze. The mouse is motivated to move from an exposed open platform to an escape hole. The mouse is trained to use visual cues to locate the escape hole as mice preferentially use visual cues to solve the maze. The arena is a circular platform with holes around the periphery. One contains an escape box that the mouse can hide in. There are spatial cues that the mouse can use to locate the escape hole. All mice have a training period for 4 days prior to the test period. Performance is measured by the latency period to escape and the number of attempts to find the escape hole measured as nose pokes in the correct hole when the box was removed.

### Fear Conditioning Response

This test assesses hippocampal function whereby a contextual fear response can be elicited when the mouse learns to associate the chamber with a mild foot shock. This is displayed as freezing behaviour. Amaygdala dependent-cued fear conditioning pairs the foot shock with a light and tone cue within a different chamber.

Mice are conditioned to a test chamber where they are later exposed to a sound followed by a foot shock. They are placed in the chamber where the length of time spent freezing is recorded (contextual freezing). On the final day, mice are placed in a different chamber, exposed to the conditioned stimulus (sound) and the time spent freezing is recorded (cued response). Freezing is defined as the complete lack of motion for a minimum of 0.75 second and the percent of freezing in each period of time are reported.

### Olfactory Test

Along with brain function and cognition, neurogenesis is known to decline with age. Age-related decline in neurogenesis is reflected in a decrease in olfactory sensitivity. To determine whether there was functional improvement following treatment with EVs, both groups of aged mice were subjected to olfactory sensitivity testing (Witt R. M. et al. J Vis Exp. 2009; (23): 949). This tests measures the ability of mice to detect reducing concentrations of an odour and finding food by odour detection. Briefly, mice are assessed against serial dilutions of a particular scent (e.g. cinnamon) and the total exploratory time at each dilution is recorded. The longer the exploratory time, the greater the olfactory sensitivity. Mice were also subjected to the “buried food test” (Dan X. et al. Ageing Res Rev. 2021; 70:101416). This test is widely used to assess general olfactory ability and integration, and measures how well a mouse detects and locates food buried under a layer of bedding. Mice are deprived of food prior to the experiment and then fed with a very small portion of food to be used in the test. A small portion of food (<2 μm.) is then buried beneath the bedding. The latency to initial digging and to finding the food is recorded. Olfactory function is assessed by these latencies wherein better function is associated with shorter time

### Results

### NORT

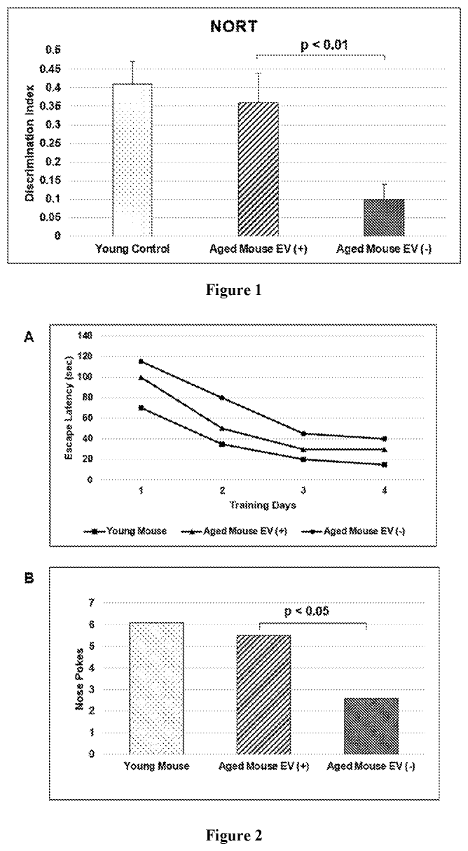

EV+ mice demonstrated a high discrimination index, similar to that observed in young control mice when exploring between the objects, as compared to EV− mice (FIG. 1; p<0.01). These data indicated an improvement in recognition memory in EV+ mice.

### Barnes Maze Test

While it was observed that mice in all treatment groups displayed improved performance in escape latency during the trial period with minimal age or treatment effect with no significant differences in latency by Day 4 (FIG. 2A), EV+ mice exhibited a similar number of attempts (nose pokes) to that observed in young control mice and had an approximate 2-fold increase in the number of nose pokes as compared to EV− mice (FIG. 2B, p<0.05). These results demonstrated enhanced learning and memory based on spatial/visual cues in EV+ mice.

### Fear Conditioning Response

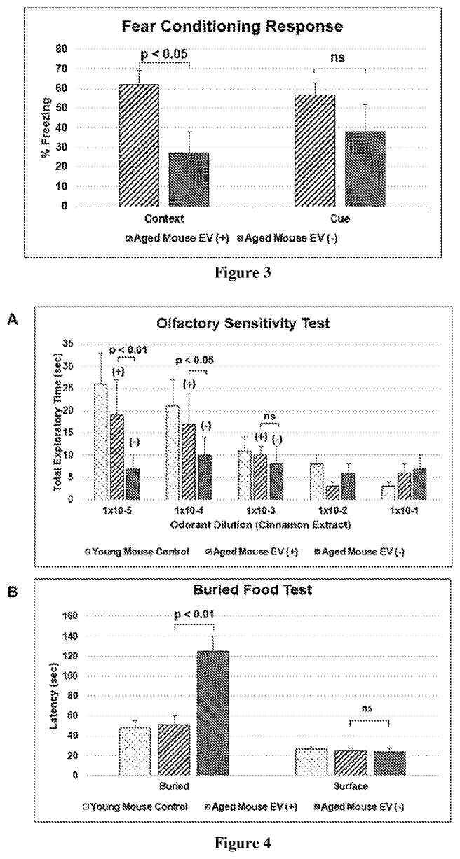

While no significant difference in the freezing time for the cued response was observed in EV+ and EV− mice, EV+ mice demonstrated a significant increase in freezing time in contextual response as compared to EV− mice (FIG. 3; p<0.05). These results indicated that EV+ mice had improved ability to learn and remember an association between environmental cues and aversive experiences.

### Olfactory Test

It was observed that EV+ and young control mice demonstrated a similar length of exploratory time at the two lowest dilutions (1×10⁻⁵ and 1×10⁻⁴) of the odorant as compared to EV− mice, however EV+ mice demonstrated a significantly longer exploratory time as compared to EV− mice (FIG. 4A; 1×10⁻⁵ p<0.01 and 1×10⁻⁴ p<0.05). Further, a shorter latency period in finding the buried food reward was also observed in EV+ mice as compared to EV− mice (FIG. 4B; p<0.01). These results suggested that EV+ mice had increased olfactory sensitivity as compared to EV− mice.

### Conclusion

EVs derived from young bovine animals are capable of increasing cognitive performance in aged mice as compared to those treated with EVs derived from old bovine animals.

### Example 2. Assessing Physical Performance in Aged Mice Treated with EVs Derived from Young and Old Bovine Animals

In this example, the inventor assessed whether treatment with EVs derived from young bovine animals improved physical performance in aged mice as compared to those treated with EVs derived from old bovine animals.

Skeletal muscle shrinks with aging in association with a decrease in the size and number of muscle fibres, which contributes to muscle strength weakening. Loss of physical performance can be observed by decreased strength in four limbs and overall physical fitness, and an increase in frailty. Several methods have been employed here to assess physical performance in aged mice following treatment with EVs derived either young or old bovine animals.

### Materials and Methods

### Tightrope Test

This assess both physical strength and motor skills. A 60 cm long rope is suspended between 2 platforms and a mouse is suspended from the middle of the rope by its forepaws and has to reach either of the platforms within a designated time. The mouse is scored based on the time taken and whether or not it succeeded in reaching the platform.

### Four Limb Hang Test

This is a test of strength and endurance where the mouse supports its weight by hanging upside down by all four limbs. Mice are adverse to falling from this position and are motivated to hold on until fatigued before falling. Mice have a high strength/weight ratio and most young mice are able to hold for the maximum time duration of the test. Here, mice are placed on a screen that is then inverted and suspended above soft bedding. The latency to fall is recorded. A maximum score is recorded when the mice does not fall within the given time frame.

### Frailty

To assess frailty, several clinical signs of deterioration/deficits were evaluated for each mouse. A score of 0 to 1 is given for each parameter, with 0=absent, 0.5=mild and 1=severe. Each mouse was weighed and its body surface temperature measured with an infrared temperature probe. Parameters scored comprised:

### Integument

Alopecia Hair loss due to age-related balding and/or barbering
Loss of fur colour or change in fur colour from black to grey or brown
Dermatitis inflammation, overgrooming, barbering or scratching causing skin erosion
Loss of whiskers due to aging and/or whisker trimming
Coat condition (ruffled fur and/or matted fur)

### Physical/Musculoskeletal

Development of tumors or masses anywhere on the body
Distended or enlarged abdomen
Kyphosis (exaggerated outward curvature of the lower cervical/thoracic vertebral column)
Tail stiffening
Gait disorder (lack of coordination in movement)
Circling or weakness
Tremor (involuntary shaking at rest or during movement)
Decline in four limb strength (falling from a suspended, inverted screen)
Body condition score (visual signs of muscle wasting or obesity based on the amount of flesh covering bony protuberances)

### Vestibulocochlear/Auditory

Vestibular disturbance (disruption in the ability to perceive motion and gravity resulting in problems with balance, orientation and acceleration
Hearing loss (failure to respond to sudden sound)

### Ocular/Nasal

Cataracts
Corneal opacity
Eye discharge/swelling
Microphthalmia
Vision loss (indicated by failure to reach toward the ground when lowered by the tail)
Menace reflex (rapid eye blink and closure of the palpebral fissure in response to a non-tactile visual threat to the eye)
Nasal discharge

### Digestive/Urogenital

Malocclusions (overgrown or uneven incisors)
Rectal prolapse
Vaginal/uterine/penile prolapse
Diarrhoea

### Respiratory

Breathing rate/depth (dyspnea, rales and/or tachypnea)

### Discomfort

Mouse grimace scale (pain/discomfort based on facial expression. Assessment of five facial features: orbital tightening, nose bulge, cheek bulge, ear position (drawn back), or whisker change (either backward or forward)
Piloerection (involuntary bristling of the fur due to sympathetic nervous system activation)

### Other

Temperature increase or decrease
Weight increase or decrease

### Results

### Tightrope Test

A significant increase in the performance score of EV+ mice similar to that in young control mice was observed as compared to EV− mice (FIG. 5; p<0.01). This demonstrated that EV+ mice had increased strength and agility as compared to EV− mice.

### Four Limb Hang Test

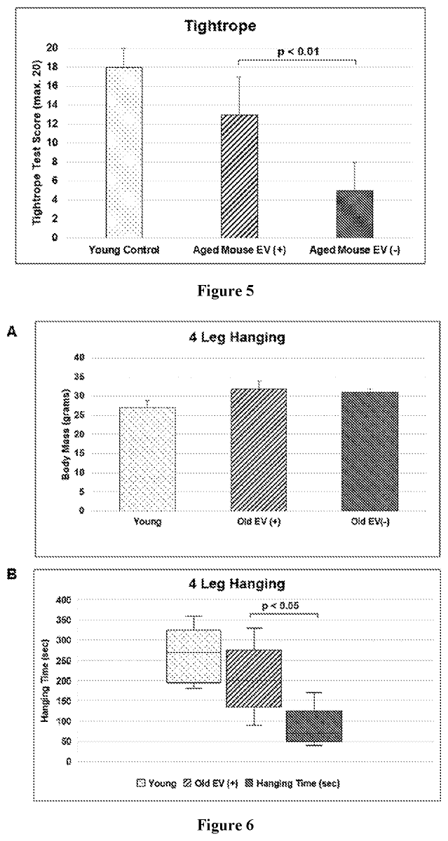

The weight of each mouse was recorded (FIG. 6A) and adjusted for when recording the elapsed time before a fall for each mouse. It was observed that EV+ mice and young control mice demonstrated a similar length of hanging time in contrast to EV− mice, who had a significantly shorter hanging time than the other two groups (FIG. 6B, p<0.05). This indicates an increased performance in strength and endurance in EV+ mice as compared to EV− mice.

### Frailty

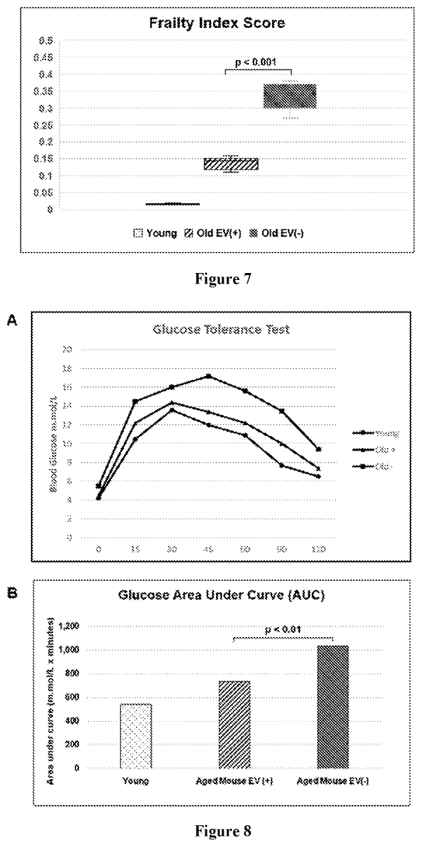

A frailty score was calculated for each mouse and plotted as median with SEM for the 3 groups. As expected, young control mice obtained a low index score of approximately 0.02. EV+ mice scored approximately 0.14 on the index, close to 2.5 fold lower on the frailty index as compared to EV− mice who scored at approximately 0.34 (FIG. 7; p<0.001). These results show that EVs from young bovine animals were able to decrease frailty in aged mice.

### Conclusion

EVs derived from young bovine animals were capable of increasing physical performance in aged mice as compared to those treated with EVs derived from old bovine animals.

### Example 3. Assessing Metabolic Function in Aged Mice Treated with EVs Derived from Young and Old Bovine Animals

In this example, the inventor assessed whether treatment with EVs derived from young bovine animals improved metabolic function in aged mice as compared to those treated with EVs derived from old bovine animals.

A common hallmark of ageing is metabolic dysfunction with one of the most involved nutrient signalling pathways being the insulin/IGF1 pathway that impacts energy homeostasis. Impaired glucose tolerance and insulin sensitivity have been observed in aged mice. Energy homeostasis is required to maintain energy balance in a steady state for optimal performance of metabolic function in an organism. Ongoing energy imbalances lead to the development of metabolic diseases such as obesity and diabetes.

The glucose tolerance test (GTT), which measures the clearance of glucose, was employed here to assess metabolic function in aged mice treated with EVs derived from either young or old bovine animals.

### Materials and Methods

Mice were fasted for 16 hours prior and baseline fasted blood glucose levels via tail vein sampling were recorded. A 20% glucose solution (2 mg/kg body weight) was injected intraperitoneally and serial blood glucose measurements using a clinical blood glucose meter were taken every 15-30 minutes over a span of 2 hours following injection to determine peak levels, clearance rates and area under the curve (AUC) to assess insulin sensitivity and glucose homeostasis.

### Results

While a similar trend in blood glucose levels following glucose administration was observed in the different treatment groups (FIG. 8A), EV− mice maintained higher blood glucose levels over a longer period of time as compared to EV+ and young control mice, suggesting an impairment or reduction of glucose clearance in these mice (FIG. 8B; p<0.01).

### Conclusion

EVs derived from young bovine animals were capable of improving metabolic function by increasing glucose sensitivity in aged mice as compared to those treated with EVs derived from old bovine animals.

### Example 4. Assessing Levels of Oxidative Stress and Inflammation in Aged Mice Treated with EVs Derived from Young and Old Bovine Animals

In this example, the inventor assessed whether treatment with EVs derived from young bovine animals improved impaired kidney and liver function in aged mice as compared to those treated with EVs derived from old bovine animals.

Chronic inflammation is known to increase with age resulting in the condition deemed “inflammaging” that is associated with age-related pathologies. Excess reactive oxygen or nitrous species (ROS/RNS) generated during inflammation can drive oxidative damage to cellular lipids and proteins. With advancing age, these processes lead to structural changes to organs such as the kidney and liver that can significantly impact their function and the overall health and lifespan of the organism. To demonstrate the effects of EVs in rejuvenating organ function, two commonly used experimental models of oxidative stress and inflammation in the kidney and liver were employed.

### Materials and Methods

### Model for Liver Injury

Prior studies have demonstrated that aged livers are more susceptible to acute liver injury than young livers. Further, the fibrotic response that emanates from the inflammatory reaction to injury is significantly greater in old mice. To mimic a model of liver injury, carbon tetrachloride (CCL4) was injected intraperitoneally (0.2 ml/kg body weight as a 20% solution in corn oil) into all three treatment groups. Mice were euthanized 96 hours following injection and several markers from serum and liver tissue were measured.

### Model for Kidney Injury

Acute kidney injury is known to pose an increased risk for the subsequent development of chronic kidney disease especially with advancing age. Haemoglobin can induce acute kidney injury, and the aged kidney is particularly sensitive to haeme proteins as demonstrated by an increase in serum creatinine, serum urea and lipid oxidation markers after exposure. The three groups of mice were administered a solution of bovine haemoglobin (2 μm/kg body weight) via the tail vein and euthanized 48 hours after. Blood and tissue were subsequently collected and parameters relevant to kidney function were measured. An additional control group comprising young mice receiving only saline was included to determine baseline values.

### Results

### Liver

Serum liver enzymes routinely used for clinical assessment of liver function were measured—serum aminotransferases (ALT, AST) and alkaline phosphatase (ALP). Also measured was the oxidative marker malondialdehyde (MDA) and degree of fibrosis (hydroxyproline).

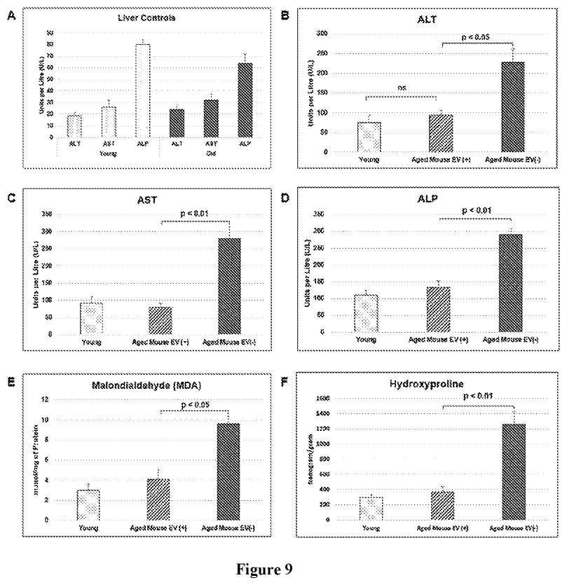

It was observed that ALT, AST and ALP levels in EV− mice remained significantly elevated after 96 hours while EV+ and young control mice (data not shown) showed declining levels of these markers, indicating resolution of oxidative stress and inflammation (FIG. 9A-D; ALT p<0.05; AST p<0.01; ALP p<0.01)). Residual lipid peroxidation, as determined by MDA detection, remained significantly higher in EV− mice as compared to EV+ and young control mice, indicating impaired clearance of and sustained oxidative damage (FIG. 9E; p<0.05). Hydroxyproline levels in EV− mice were also significantly higher as compared to EV+ and young control mice, indicating a prolonged fibrotic response in these mice (FIG. 9F; p<0.01).

### Kidney

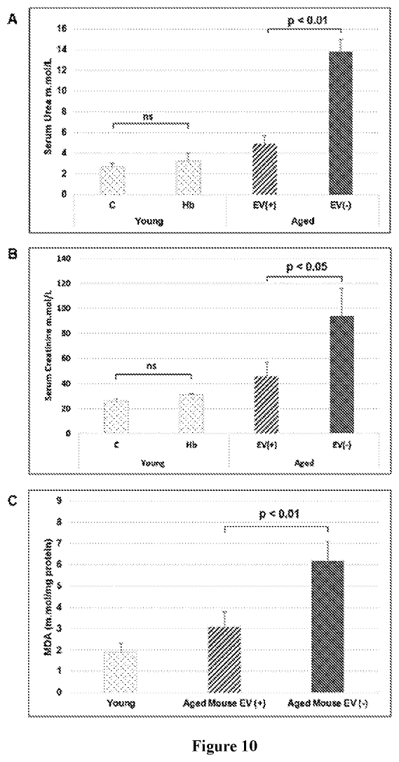

No differences were observed between EV+ and young control mice however EV− mice demonstrated a significant, sustained increase in serum urea and serum creatinine 48 hrs following haemoglobin administration as compared to EV+ and young control mice (FIGS. 10A and B; p<0.01 and p<0.05 respectively), indicating a slower resolution of the acute insult in these mice. MDA levels also persisted in EV− mice over the time course as compared to the other two treatment groups, indicating sustained oxidative damage to the kidneys of these mice (FIG. 10C; p<0.01).

### Conclusion

EVs derived from young bovine animals were capable of reducing oxidative stress- and inflammation induced-damage in aged mice as compared to those treated with EVs derived from old bovine animals.

1. An oral composition comprising a therapeutically effective amount of extracellular vesicles (EVs) derived from blood, a blood fraction and/or tissue of a young, non-human animal.

2. The composition of claim 1 wherein the young, non-human animal has not yet reached sexual maturity.

3. The composition of claim 1 or claim 2 wherein the EVs are selected from the group consisting of exosomes, microvesicles, membrane particles, membrane vesicles, exosome-like vesicles, extracellular vesicles, exovesicles and combinations thereof.

4. The composition of any one of claims 1 to 3, wherein the EVs are derived from a livestock species.

5. The composition of any one of claims 1 to 4, wherein the EVs are derived from a non-human mammal.

6. The composition of claim 5, wherein the non-human mammal is selected from a bovine, ovine, equine, caprine, porcine or camelid.

7. The composition of any one of claims 1 to 4, wherein the EVs are derived from an avian livestock species.

8. The composition of any one of claims 1 to 7, wherein the EVs are derived from whole blood, serum, plasma and/or bone marrow.

9. The composition of any one of claims 1 to 7, wherein the EVs are derived from at least one tissue selected from the group consisting of adipose tissue, thymus, pancreas, lung, heart, liver, muscle, intestine and combinations thereof.

10. The composition of any one of claims 1 to 7 and 9, wherein the EVs are derived from adipose tissue.

11. The composition of any one of claims 1 to 10, wherein the EVs are present in the composition at a concentration which is higher than is present in the blood, blood fraction or tissue from which they are derived.

12. The composition of any one of claims 1 to 11, wherein the EVs have been purified.

13. The composition of any one of claims 1 to 12, wherein the EVs are lyophilized, spray-dried or gelated.

14. The composition of any one of claims 1 to 13, wherein the EVs are encapsulated.

15. The composition of any one of claims 1 to 13, wherein the EVs are provided within a capsule.

16. The composition of any one of claims 1 to 12, wherein the EVs are provided in a liquid form.

17. A method of preparing a composition of any one of claims 1 to 16, comprising:
a. obtaining blood, a blood fraction and/or tissue from a non-human mammal; and
b. isolating the EVs from one or more other components of the blood, blood fraction and/or tissue.

18. The method of claim 17, further comprising performing one or more purification processes and/or one of more processes to concentrate the EVs.

19. The method of claim 18, wherein the one or more purification processes are selected from size exclusion chromatography, ultracentrifugation, polymer or calcium ion or salt-induced precipitation and ultrafiltration.

20. The method of claim 18, wherein the EVs are concentrated by ultrafiltration and centrifugation.

21. The method of any one of claims 17 to 20, further comprises lyophilizing the EVs.

22. A composition when produced by the method of any one of claims 17 to 21.

23. A method of treating, preventing or delaying an age-related condition or pathology in a subject, comprising orally administering to the subject the composition of any one of claim 1 to 16 or 22.

24. Use of a composition of any one of claims 1 to 16 in the manufacture of a medicament for treating, preventing or delaying an age-related condition or pathology in a subject.

25. The method of claim 23 or the use of claim 24, wherein the age-related condition or pathology is associated with a decline in one or more of cognitive function, physical performance and/or metabolic function.

26. The method or use of claim 25, wherein a decline in cognitive function comprises a decline in one or more of alertness, executive function, memory, learning, visual-spacial processing utilizing the frontal cortex, thalamus, dorso-lateral prefrontal cortex and/or hippocampus, and/or olfactory sensitivity.

27. The method or use of claim 25 or 26, wherein a decline in physical performance comprises a decline in one or more of musculoskeletal strength, motor skills and/or balance, and/or an increase in frailty.

28. The method or use of any one of claims 25 to 27, wherein a decline in metabolic function comprises impaired glucose tolerance and/or insulin insensitivity.

29. The method of claim 23 or the use of claim 24, wherein the age-related condition or pathology is associated with inflammation and/or oxidative stress.

30. The method or use of claim 29, wherein the inflammation and/or oxidative stress is associated with impaired kidney function, acute kidney injury, chronic kidney disease, or acute and chronic liver injury in the subject.

31. A method of improving kidney and/or liver function in a subject suffering from a kidney and/or liver condition, comprising orally administering to the subject the composition of any one of claim 1 to 16 or 22.

32. Use of a composition of any one of claims 1 to 16 in the manufacture of a medicament for improving kidney and/or liver function in a subject suffering from a kidney and/or liver condition.

33. The method of claim 31 or the use of claim 32, wherein the kidney condition is an acute kidney injury and/or the liver condition is acute liver injury.

34. The method of claim 31 or the use of claim 32, wherein the kidney condition is a chronic kidney disease and/or the liver condition is chronic liver disease.

35. A method of improving a decline in one or more of cognitive function, physical performance and/or metabolic function by orally administrating a composition of any one of claims 1 to 16 to a subject in need thereof.
